(19) United States
(12) Patent Application Publication
(10) Pub. No.: US 2026/0144756 A1
(43) Pub. Date: May 28, 2026

(54) A FENOFIBRATE COMPOSITION COMPRISING WATER SOLUBLE CHEMICALS AND A METHOD OF PRODUCTION THEREOF

(71) Applicant: US NANO FOOD AND DRUG INC, Wilmington, DE (US)

(72) Inventors: Yip Ching PUI, Sydney, New South Wales (AU); Yip Shu PUI, Sydney, New South Wales (AU); Hing Sang PUI, Sydney, New South Wales (AU)

(21) Appl. No.: 19/122,801

(22) PCT Filed: Oct. 20, 2023
(86) PCT No.: PCT/IB2023/060598

§ 371 Date: Apr. 21, 2025

(30) Foreign Application Priority Data
Oct. 20, 2022 (AU) 2022903094

(51) Int. Cl. A61K 9/20; A61K 31/216

(52) U.S. Cl. CPC A61K 9/2095; A61K 9/2013; A61K 9/2027; A61K 9/2054; A61K 31/216

(57) ABSTRACT

A fenofibrate composition comprising water soluble chemicals, the composition having a higher dissolution rate in an aqueous medium, wherein the composition is prepared by mixing fenofibrate in an organic solvent solution with an organic acid and other excipients to produce the composition in the form of granules, a tablet or a capsule.

### CROSS REFERENCE TO RELATED APPLICATIONS

This application claims priority to Australian Provisional Patent Application No. 2022903094 filed 20 Oct. 2022, which is incorporated herein by cross-reference in its entirety as if set forth in full.

### FIELD OF THE INVENTION

The present invention broadly relates to a method of producing an oral composition of fenofibrate using an organic solvent solution and formulations thereof, wherein the composition of fenofibrate has a higher dissolution rate in aqueous medium. The composition of the invention may in the form of a tablet, a capsule, or granules.

### BACKGROUND

Fenofibrate is a lipid-lowering fibrate used in the management of primary and secondary hyperlipidaemia, when there is a lack of clinical improvement following lifestyle modifications or correction of the underlying cause. There are many strengths of fenofibrate on the market formulated for different modes of administration, however all are produced using the same method which involves mechanical micronization. Fenofibrate is regarded as a prodrug and is metabolised in vivo to its active metabolite fenofibric acid. The elimination half-life of fenofibric acid is about 20 hours.

The chemical name of fenofibrate is (2-(4-(4-chlorobenzoyl)phenoxy)-2-methylpropanoic acid 1-methyl ethyl ester or propan-2-yl 2-[4-(4-chlorobenzoyl)phenoxy]-2-methylpropanoate). Fenofibric acid is effective in reducing total cholesterol, LDL cholesterol, and total triglycerides. The first product on the market was a 100 mg fenofibrate tablet to be administered three times daily. The second product was a 200 mg capsule to be administered once daily. The third product was a 160 mg fenofibrate tablet to be administered once daily. The fourth product was a 145 mg tablet to be administered once daily. The fifth product was a 130 mg capsule to be administered once daily. The sixth product was a 90 mg fenofibrate capsule to be administered once daily.

Fenofibrate is water insoluble and is poorly absorbed when administered orally, consequently high oral doses are often required in order for the plasma concentration of fenofibric acid to reach a therapeutically effective level. This leads to a high concentration of fenofibrate in the alimentary system which induces side effects. The poor absorption leads to inadequate and variable bioavailability and gastrointestinal mucosal toxicity.

The absorption of a drug is often related to the particle size of the drug and as a particle becomes smaller, the surface area to volume ratio increases allowing greater solvent interaction which increases solubility. Particle size reduction is therefore a commonly used method to increase solubility and is often achieved by drug dispersion in carriers such as solid dispersions, solid solutions, as well as by cryogenic techniques and co-micronization.

There is a need to develop a new product with high dissolution rate of fenofibrate in the gastrointestinal system to provide the same therapeutic concentration as the 200 mg fenofibrate composition but with a smaller daily dosage such as 160 mg per tablet, 90 mg per tablet, 85 mg per tablet or 80 mg per tablet.

There are many prior art compositions of fenofibrate in the world as follows:
U.S. Pat. No. 5,145,684 discloses nanoparticles of drug substances including lipid regulating agents having a non-crosslinked surface modifier.
U.S. Pat. Nos. 6,375,986; 6,969,529; 7,320,802; 7,276,249; 7,927,627; 6,592,903 and US patent applications 2004/0087656; 2008/0241070; 2008/0138424 and 2007/0264348 disclose nanoparticulate compositions of fenofibrate.
U.S. Pat. Nos. 6,277,405; 6,074,670; 6,652,881; 7,037,529; 7,041,319; 6,589,552; 6,531,158; 7,101,574 and 7,863,331 and US patent applications 2004/0057998; 2007/0071812; 2008/0248101; 2011/0159082; 2010/0112049 and 2009/0035379 describe micronized fenofibrate compositions.
U.S. Pat. Nos. 4,895,726; 5,880,148 and 7,189,412 describe compositions wherein fenofibrate is co-micronized with surface-active agents.
U.S. Pat. No. 7,658,944 discloses tablet compositions of fenofibrate with PEG and Poloxamer. U.S. Pat. No. 7,976,869 discloses fenofibrate tablets and granules comprising micronized fenofibrate, polyvinylpyrrolidone, and pregelatinised starch.
U.S. Pat. No. 6,828,334 discloses inclusion complexes of fenofibrate with cyclodextrins.
U.S. Pat. No. 6,027,747 discloses solid dispersions of fenofibrate.
US 2006/0222706 and US 2006/0222707 describe fenofibrate in intimate association with menthol and surfactant active agents which provides enhanced bioavailability of fenofibrate.
WO 2010/082214 discloses a fenofibrate formulation with enhanced oral bioavailability comprising fenofibrate dissolved in a lipophilic surfactant. It also discloses that such formulations at lower doses may improve side effect profile.
US 2007/0014846 discloses compositions, particularly pharmaceutical compositions in particulate form, such as granulated or solid dosage forms comprising a combination of a fibrate and a statin. More specifically, it discloses a solid pharmaceutical composition comprising atorvastatin and a low dose, i.e., a reduced amount of fenofibrate having improved bioavailability and/or improved pharmacological response, i.e., providing an improved effect.
US 2004/0057999 discloses an orally administrable fenofibrate tablet, wherein the required daily dose is lower than 200 mg.

The present invention relates to a reduced dose composition of fenofibrate.

The reduced dose composition of the present invention offers advantages that may include, but not limited to, one or more of: (1) smaller doses of drug required to obtain the same pharmacological effect; (2) substantially similar pharmacokinetic profiles of fenofibric acid resulting from the fenofibrate composition when administered in the fed and the fasted state; and (3) reduction of side-effects associated with higher doses.

### SUMMARY

According to a first aspect, the present invention relates a pharmaceutical composition of fenofibrate comprising fenofibrate, a water soluble excipient selected from a group comprising an organic acid which is selected from the group comprising malic acid, maleic acid, succinic acid, tartaric acid, citric acid and ascorbic acid, hydroxypropyl methyl cellulose (HPMC), hydroxypropyl cellulose, and wherein the composition is produced by dissolving the fenofibrate and the water soluble excipient in ketone which is mixed with an expander and filler, wherein the composition produced is in the form of pellets, capsules, granules or tablets.

In one embodiment the pharmaceutical composition comprises fenofibrate, a water soluble excipient selected from a group comprising an organic acid, wherein the organic acid is selected from the group comprising malic acid, maleic acid, succinic acid, tartaric acid, citric acid and ascorbic acid; hydroxypropyl methyl cellulose (HPMC) and/or hydroxypropyl cellulose, and wherein the composition is produced by dissolving the fenofibrate and the water soluble excipient in ketone which is subsequently mixed with an expander and filler; wherein the composition produced is in the form of pellets, capsules, granules or tablets.

Preferably, the expander and filler of the composition comprises cross-linked povidone, cross-linked carboxymethyl sodium cellulose, lactose, sugar, microcrystalline cellulose, a small amount of povidone and sodium dodecyl sulfate, wherein the composition is in the form of granules, tablets or capsules.

Preferably, the fenofibrate composition comprises 1 part of fenofibrate and 1 to 6 parts of an organic acid which is selected from the group comprising malic acid, tartaric acid, maleic acid, succinic acid, fumaric acid, ascorbic acid and citric acid, wherein the composition is in the form of granules, tablets or capsules.

Preferably, the fenofibrate composition comprises fenofibrate, an organic acid selected from the group comprising malic acid, tartaric acid, maleic acid, succinic acid, ascorbic acid and citric acid, wherein the composition further comprises a carbonate salt selected from calcium carbonate, sodium bicarbonate and potassium bicarbonate, and wherein the composition is in the form of tablets.

Preferably, the fenofibrate composition comprises 1 part of fenofibrate and 1 to 6 parts of an organic acid which is selected from the group comprising malic acid, tartaric acid, maleic acid, succinic acid, ascorbic acid and citric acid; wherein the composition further comprises cross-linked povidone, cross-linked carboxymethyl sodium cellulose and sodium dodecyl sulfate, wherein the composition is in the form of granules, tablets or capsules.

Preferably, the composition comprises fenofibrate, a water soluble excipient including an organic acid selected from the group comprising malic acid, maleic acid, succinic acid, tartaric acid, ascorbic acid and citric acid, wherein the composition further comprises HPMC, HPC, cross-linked povidone and cross-linked carboxymethyl sodium cellulose.

In one or more embodiments, the pharmaceutical composition comprises 1 part of fenofibrate and 1 to 6 parts of malic acid, or 1 part of fenofibrate and 1 to 6 parts of maleic acid, or 1 part of fenofibrate and 1 to 6 parts of tartaric acid, or 1 part of fenofibrate and 1 to 6 parts of citric acid, or 1 part of fenofibrate and 1 to 6 parts of succinic acid.

Preferably, the composition comprises fenofibrate, malic acid, cross-linked povidone, cross-linked carboxymethyl sodium cellulose, wherein the composition is in the form of granules, tablets or capsules.

Preferably, the composition comprises fenofibrate, maleic acid, cross-linked povidone, cross-linked carboxymethyl sodium cellulose, wherein the composition is in the form of granules, tablets or capsules.

Preferably, the composition comprises fenofibrate, tartaric acid, cross-linked povidone, cross-linked carboxymethyl sodium cellulose, wherein the composition is in the form of granules, tablets or capsules.

Preferably, the composition comprises fenofibrate, succinic acid, cross-linked povidone, cross-linked carboxymethyl sodium cellulose, wherein the composition is in the form of granules, tablets or capsules.

Preferably, the composition comprises fenofibrate, citric acid, cross-linked povidone, cross-linked carboxymethyl sodium cellulose, wherein the composition is in the form of granules, tablets or capsules.

Preferably, the composition comprises fenofibrate, an organic acid which is selected from the group comprising malic acid, tartaric acid, maleic acid, succinic acid, fumaric acid, and citric acid, HPMC, HPC and a core comprising sugar and/or microcrystalline cellulose, wherein the composition is in the form of spheres.

Preferably, the composition comprises fenofibrate, an organic acid which is selected from the group comprising malic acid, tartaric acid, maleic acid, succinic acid, fumaric acid, and citric acid, HPC and a core comprising sugar and/or microcrystalline cellulose, wherein the composition is in the form of spheres.

Preferably, the composition comprises fenofibrate, an organic acid which is selected from the group comprising malic acid, tartaric acid, maleic acid, succinic acid, fumaric acid, and citric acid, HPMC and a core comprising sucrose and/or microcrystalline cellulose, wherein the composition is in the form of spheres.

Preferably, the composition comprises fenofibrate, an organic acid which is selected from the group comprising malic acid, tartaric acid, maleic acid, succinic acid and citric acid, calcium carbonate, cross-linked povidone, cross-linked carboxymethyl sodium cellulose, wherein the composition is in the form of tablets.

According to a second aspect, the present invention relates a method of preparing a fenofibrate composition comprising the steps of:
a) forming a fenofibrate solution by dissolving an amount of fenofibrate, hydroxypropyl methyl cellulose (HPMC), and hydroxypropyl cellulose (HPC) in an organic solvent, wherein the solvent is selected from acetone, butanone or pentanone;
b) optionally dissolving or dispersing an organic acid into the fenofibrate solution of step a), wherein the organic acid is selected from the group comprising malic acid, maleic acid, succinic acid, tartaric acid, fumaric acid, ascorbic acid and citric acid;
c) spraying the fenofibrate solution or dispersion of step a) or b) into a fluid bed or granulator containing an expander and filler to form a powder mixture;
d) the powder mixture of step c) is glued together to form granules using povidone (PVPK30), HPMC or HPC, and the granules are dried in the fluid bed or an oven; and
e) the granules of step d) are compressed into a tablet or filled into a capsule or a granules bag.

In one embodiment, the fenofibrate composition produced by the method of the second aspect, is the fenofibrate composition of the first aspect.

Preferably, the expander and filler of the composition comprises cross-linked povidone, cross-linked carboxymethyl sodium cellulose, lactose, sucrose, a small amount of povidone and sodium dodecyl sulfate, wherein the composition is in the form of granules, tablets or capsules.

Preferably, the fenofibrate composition comprises 1 part of fenofibrate and 1 to 6 parts of organic acid which is selected from the group comprising malic acid, tartaric acid, maleic acid, succinic acid, fumaric acid, ascorbic acid and citric acid, wherein the composition is in the form of granules, tablets or capsules.

Preferably, the fenofibrate composition comprises 1 part of fenofibrate and 1 to 6 parts of an organic acid which is selected from the group comprising malic acid, tartaric acid, maleic acid, succinic acid, fumaric acid, ascorbic acid and citric acid; wherein the composition further comprises cross-linked povidone, cross-linked carboxymethyl sodium cellulose, a small amount of povidone and sodium dodecyl sulfate, wherein the composition is in the form of granules, tablets or capsules.

Another method of producing a fenofibrate composition of the invention comprises the steps of:
a) suspending an amount of fenofibrate in water;
b) dissolving a water soluble organic acid selected from the group comprising malic acid, maleic acid, succinic acid, tartaric acid, citric acid and ascorbic acid, into the fenofibrate suspension of step a);
c) homogenizing the mixture of step b) using a grinding machine 5 to 10 times to make a mixture of uniform dispersion;
d) spraying the fenofibrate mixture of step c) into a fluid bed or granulator containing an expander and filler to form a powder mixture which is glued together to form granules using povidone (PVPK30); and
e) compressing the dried granules into a tablet or filling the dried granules into a capsules.

Preferably, the composition is in the form of granules, tablets or capsules.

Preferably, the fenofibrate organic acid composition comprises 1 part of fenofibrate and 1 to 6 parts of organic acid which is selected from the group comprising malic acid, tartaric acid, maleic acid, succinic acid, fumaric acid, ascorbic acid and citric acid, wherein the composition is in the form of granules, tablets or capsules.

Preferably, the fenofibrate composition comprises 1 part of fenofibrate and 1 to 6 parts of an organic acid which is selected from the group comprising malic acid, tartaric acid, maleic acid, succinic acid, fumaric acid, ascorbic acid and citric acid; wherein the composition further comprises cross-linked povidone, cross-linked carboxymethyl sodium cellulose, a small amount of povidone and sodium dodecyl sulfate, wherein the composition is in the form of granules, tablets or capsules.

Preferably, the fenofibrate composition comprises fenofibrate, malic acid, cross-linked povidone, cross-linked carboxymethyl sodium cellulose, a small amount of povidone and sodium dodecyl sulfate, wherein the composition is in the form of granules, tablets or capsules.

Preferably, the fenofibrate composition comprises fenofibrate, maleic acid, cross-linked povidone, cross-linked carboxymethyl sodium cellulose, a small amount of povidone and sodium dodecyl sulfate, wherein the composition is in the form of granules, tablets or capsules.

Preferably, the fenofibrate composition comprises fenofibrate, tartaric acid, cross-linked povidone, cross-linked carboxymethyl sodium cellulose, a small amount of povidone and sodium dodecyl sulfate, wherein the composition is in the form of granules, tablets or capsules.

Preferably, the fenofibrate composition comprises fenofibrate, succinic acid, cross-linked povidone, cross-linked carboxymethyl sodium cellulose, a small amount of povidone and sodium dodecyl sulfate, wherein the composition is in the form of granules, tablets or capsules.

Preferably, the fenofibrate composition comprises fenofibrate, citric acid, cross-linked povidone, cross-linked carboxymethyl sodium cellulose, a small amount of povidone and sodium dodecyl sulfate, wherein the composition is in the form of granules, tablets or capsules.

Preferably, the fenofibrate composition comprises fenofibrate, ascorbic acid, cross-linked povidone, cross-linked carboxymethyl sodium cellulose, a small amount of povidone and sodium dodecyl sulfate, wherein the composition is in the form of granules, tablets or capsules.

In another aspect, the present invention provides reduced dose composition of fenofibrate, comprising fenofibrate, a water soluble excipient including an organic acid selected from the group comprising malic acid, maleic acid, succinic acid, tartaric acid, ascorbic acid and citric acid, wherein the composition further comprises cross-linked povidone and cross-linked carboxymethyl sodium cellulose.

Preferably, the composition comprises 1 part of fenofibrate and 1 to 6 parts of organic acid selected from the group comprising malic acid, maleic acid, succinic acid, tartaric acid, ascorbic acid and citric acid, the composition further comprising cross-linked povidone, cross-linked carboxymethyl sodium cellulose, a small amount of sodium dodecyl sulfate, and wherein the composition is in the form of granules, tablets or capsules.

Preferably, the reduced dose composition comprises fenofibrate and an organic acid selected from the group comprising malic acid, maleic acid, succinic acid, tartaric acid, ascorbic acid and citric acid, the composition further comprising cross-linked povidone, cross-linked carboxymethyl sodium cellulose, a small amount of sodium dodecyl sulfate, and wherein the composition is in the form of granules, tablets or capsules.

Preferably, the composition comprises 1 part of fenofibrate and 1 to 6 parts of organic acid.

Preferably, the fenofibrate composition comprises fenofibrate, organic acid, cross-linked povidone, cross-linked carboxymethyl sodium cellulose, a small amount of povidone and sodium dodecyl sulfate, wherein the composition is in the form of granules, tablets or capsules.

Preferably, the fenofibrate composition comprises 1 part of fenofibrate, 6 parts of organic acid, cross-linked povidone, cross-linked carboxymethyl sodium cellulose, a small amount of povidone and sodium dodecyl sulfate, wherein the composition is in the form of granules, tablets or capsules

Preferably, wherein the organic acid is malic acid.

Preferably, wherein the organic acid is maleic acid.

Preferably, wherein the organic acid is tartaric acid.

Preferably, wherein the organic acid is citric acid.

Preferably, wherein the organic acid is succinic acid.

Preferably, wherein the organic acid is ascorbic acid.

Preferably, wherein the organic acid is fumaric acid.

Preferably, the fenofibrate composition comprises 1 part of fenofibrate, 1 to 6 parts of a water soluble organic acid selected from the group comprising malic acid, maleic acid, succinic acid, tartaric acid, fumaric acid, ascorbic acid and citric acid, wherein the composition further comprises one or more excipients selected from cross-linked povidone, cross-linked carboxymethyl sodium cellulose, lactose and a small amount of SDS wherein the composition is in the form of granules, capsules, or tablets.

Preferably, the fenofibrate composition comprises 1 part of fenofibrate, 1 to 6 parts of malic acid, 1 to 2 parts of cross-linked povidone, 1 to 2 parts of cross-linked carboxymethyl sodium cellulose, 1 to 2 parts of lactose and a small amount of SDS, wherein the composition is in the form of granules, capsules, or tablets.

Preferably, the fenofibrate composition comprises 1 part of fenofibrate, 1 to 6 parts of maleic acid, 1 to 2 parts of cross-linked povidone, 1 to 2 parts of cross-linked carboxymethyl sodium cellulose, 1 to 2 parts of lactose and a small amount of SDS, wherein the composition is in the form of granules, capsules, or tablets.

Preferably, the fenofibrate composition comprises 1 part of fenofibrate, 1 to 6 parts of succinic acid, 1 to 2 parts of cross-linked povidone, 1 to 2 parts of cross-linked carboxymethyl sodium cellulose, 1 to 2 parts of lactose and a small amount of SDS, wherein the composition is in the form of granules, capsules, or tablets.

Preferably, the fenofibrate composition comprises 1 part of fenofibrate, 1 to 6 parts of tartaric acid, 1 to 2 parts of cross-linked povidone, 1 to 2 parts of cross-linked carboxymethyl sodium cellulose, 1 to 2 parts of lactose and a small amount of SDS, wherein the composition is in the form of granules, capsules, or tablets.

Preferably, the fenofibrate composition comprises 1 part of fenofibrate, 1 to 6 parts of citric acid, 1 to 2 parts of cross-linked povidone, 1 to 2 parts of cross-linked carboxymethyl sodium cellulose, 1 to 2 parts of lactose and a small amount of SDS, wherein the composition is in the form of granules, capsules, or tablets.

Preferably, the reduced dose composition comprises fenofibrate and an organic acid selected from the group comprising malic acid, maleic acid, succinic acid, tartaric acid, fumaric acid and citric acid, the composition further comprising cross-linked povidone, HPMC, HPC, cross-linked carboxymethyl sodium cellulose, a small amount of sodium dodecyl sulfate, sucrose, microcrystalline cellulose, and wherein the composition is in the form of granules, sphere, tablets or capsules.

Preferably, the reduced dose composition comprises fenofibrate, an organic acid selected from the group comprising malic acid, maleic acid, succinic acid, tartaric acid, fumaric acid and citric acid, a carbonate salt selected from calcium carbonate, potassium bicarbonate or sodium bicarbonate, the composition further comprising cross-linked povidone, cross-linked carboxymethyl sodium, HPMC, cellulose, and wherein the composition is in the form of granules, tablets or capsules.

Preferably, the reduced dose composition comprises fenofibrate, an organic acid selected from the group comprising malic acid, maleic acid, succinic acid, tartaric acid and citric acid, calcium carbonate, the composition further comprising cross-linked povidone, cross-linked carboxymethyl sodium cellulose, HPMC, and wherein the composition is in the form of granules or dispersible tablets.

Preferably, the reduced dose composition comprises fenofibrate, an organic acid selected from the group comprising malic acid, maleic acid, succinic acid, tartaric acid and citric acid, sodium bicarbonate, the composition further comprising cross-linked povidone, cross-linked carboxymethyl sodium cellulose, HPMC, and wherein the composition is in the form of granules or dispersible tablets.

Preferably, the reduced dose composition comprises fenofibrate, an organic acid, HPMC of low molecular weight, a core comprising sucrose and/or microcrystalline cellulose, and wherein the composition is in the form of spheres.

Preferably, the reduced dose composition comprises fenofibrate, an organic acid selected from the group comprising malic acid, maleic acid, succinic acid, tartaric acid and citric acid, HPMC of low molecular weight, a core comprising sucrose and/or microcrystalline cellulose, and wherein the composition is in the form of spheres or capsules.

Preferably, the reduced dose composition comprises fenofibrate, an organic acid selected from the group comprising malic acid, maleic acid, succinic acid, tartaric acid, fumaric acid and citric acid, hydroxypropyl cellulose of low molecular weight, a core comprising sucrose and/or microcrystalline cellulose and wherein the composition is in the form of spheres.

In preferred embodiments, the composition is in the form of spheres comprising micronized fenofibrate, an organic acid, HPMC or HPC of low molecular weight, a core comprising microcrystalline cellulose and/or sucrose, and wherein the composition is in the form of spheres.

In preferred embodiments, the composition is in the form of spheres comprising micronized fenofibrate, an organic acid, HPC of low molecular weight, a core comprising microcrystalline cellulose and/or sucrose, and wherein the composition is in the form of spheres.

In preferred embodiments, the composition is in the form of spheres comprising micronized fenofibrate, an organic acid, HPMC of low molecular weight, a core comprising microcrystalline cellulose and/or sucrose, and wherein the composition is in the form of spheres.

In preferred embodiments, the HPMC is of a molecular weight equal to or lower than 26,000 g/mol.

In another aspect, the present invention provides a method of treating hyperlipidemia or hypercholesterolemia in a patient, comprising administering to the patient the composition produced by the method as described herein, or the composition as described herein, wherein less than 160 mg of fenofibrate is administered to the patient once daily, or wherein less than 85 mg fenofibrate is administered to the patient once daily.

In another aspect, the present invention provides use of the composition produced by the method as described herein, or the composition as described herein, in the manufacture of a medicament for treating hyperlipidemia or hypercholesterolemia in a patient, wherein less than 160 mg of fenofibrate is administered to the patient once daily, or wherein less than 85 mg fenofibrate is to be administered to the patient once daily.

In another aspect, the present invention provides use of the composition produced by the method as described herein, or the composition as described herein, in the manufacture of a medicament for treating hyperlipidemia or hypercholesterolemia in a patient. Preferably, wherein less than or equal to 160 mg fenofibrate is to be administered to the patient once daily, or wherein less than 85 mg fenofibrate is to be administered to the patient once daily.

In another aspect, the present invention provides use of the composition produced by the method as described herein, or the reduced dose composition as described herein, in the manufacture of a medicament for treating hyperlipidemia or hypercholesterolemia in a patient, wherein less than 145 mg fenofibrate is to be administered to the patient once daily.

In another aspect, the present invention provides the composition produced by the method as described herein, or the reduced dose composition as described herein, for use in treating hyperlipidemia or hypercholesterolemia.

In one embodiment, the present invention relates to a reduced dose composition of fenofibrate comprising micronized fenofibrate, a water soluble organic acid which is selected from the group comprising malic acid, maleic acid, succinic acid, tartaric acid, ascorbic acid and citric acid, and a carbonate salt selected from calcium carbonate and sodium bicarbonate, to produce a tablet which has a high dissolution rate of fenofibrate and fast disintegration.

In one embodiment, the present invention is made by dissolving an amount of fenofibrate in an organic solvent selected from a group consisting of a ketone such as acetone, butanone and pentanone, dichloromethane, trichloromethane, and an alkanol such as ethanol and isopropyl alcohol; the fenofibrate solution is sprayed into a fluid bed or wet granulator containing cross-linked povidone, cross-linked carboxymethyl sodium cellulose, sucrose and lactose; and the granular powder formed is glued together by aqueous povidone (PVPK30) to form granules which are filled into a capsule or compressed into a tablet.

In another method of production of the invention, a certain amount of an organic acid selected from the group comprising malic acid, maleic acid, succinic acid, tartaric acid and citric acid, is dissolved in the fenofibrate organic solvent solution as described herein; the resulting fenofibrate and organic acid solution is then sprayed into a fluid bed or wet granulator containing cross-linked povidone, cross-linked carboxymethyl sodium cellulose, sucrose and lactose; and the powder formed is glued together by aqueous povidone (PVPK30) to form granules which are filled into a capsule or compressed into a tablet.

In one embodiment, the composition of the fenofibrate of the invention comprises 1 part of fenofibrate, 2 to 3 parts of cross-linked povidone, 2 to 3 parts of cross-linked carboxymethyl sodium cellulose and 1 to 2 parts of sucrose and/or lactose by weight; the fenofibrate dissolution rate of the composition of the invention is about the same as the fenofibrate dissolution rate of a LIPANTHYL™ 160 mg tablet. The fenofibrate of the LIPANTHYL™ 160 mg tablet is made with micronized raw material.

In one embodiment, a composition of the invention comprises 145 mg fenofibrate, the composition comprising 1 part of fenofibrate, 1 part of an organic acid selected from the group comprising malic acid, maleic acid, succinic acid, tartaric acid and citric acid, ascorbic acid, about 1 part of cross-linked povidone, and 1 part of cross-linked carboxymethyl sodium cellulose by weight; the fenofibrate dissolution rate of the 145 mg fenofibrate composition of the invention is about the same as the fenofibrate dissolution rate of a LIPANTHYL™ 160 mg tablet. The fenofibrate of the LIPANTHYL™ 160 mg tablet is made with micronized raw material.

In one embodiment, a composition of the invention comprises 1 part of fenofibrate, 1 to 6 parts of an organic acid selected from the group comprising malic acid, maleic acid, succinic acid, tartaric acid, fumaric acid and citric acid, about 1 part of cross-linked povidone, 1 part of cross-linked carboxymethyl sodium cellulose by weight; the fenofibrate dissolution rate of the composition of the invention containing the organic acid is higher than the fenofibrate dissolution rate of the LIPANTHYL™ 160 mg tablet, although the fenofibrate of the LIPANTHYL™ 160 mg tablet is made with micronized raw material.

One embodiment of the present invention relates to a reduced dose composition of fenofibrate made by an organic solvent solution containing fenofibrate and cross-linked povidone, cross-linked carboxymethyl sodium cellulose and lactose, having improved dissolution properties, wherein the organic solvent may be a ketone. Preferably, the embodiment of the invention comprises about 160 mg of fenofibrate per dosage and has a dissolution of from about 55 mg to 105 mg of fenofibrate in 1000 mL of neutral pure water containing 2.5 g of sodium dodecyl sulphate when stirred with a rotation speed of 75 RPM at 60 minutes. Preferably, the composition is administered once daily.

Another embodiment relates to a reduced dose composition of fenofibrate made by an organic solvent solution containing 1 part of fenofibrate and 1 to 6 parts by weight of an organic acid selected from the group comprising malic acid, tartaric acid, maleic acid, succinic acid and citric acid; wherein the amount of organic solvent is 2 to 4 times the amount of organic acid, wherein the organic solvent is selected from a group consisting of a ketone such as acetone, butanone and pentanone, dichloromethane, trichloromethane, and an alkanol such as ethanol and isopropyl alcohol, or a mixture thereof. Preferably, the composition further comprises cross-linked povidone, cross-linked carboxymethyl sodium cellulose and lactose having improved dissolution properties. Preferably, the composition is administered once daily.

Another embodiment of the composition of the invention comprises 1 part of fenofibrate and 1 part of organic acid by weight, wherein the organic acid is selected from the group comprising malic acid, tartaric acid, maleic acid, succinic acid, fumaric acid and citric acid, and one or more pharmaceutically acceptable excipients. Preferably in this composition, the amount of fenofibrate is 145 mg per dosage which exhibits a dissolution of 55 mg of fenofibrate in 1000 mL of neutral pure water containing 2.5 g of sodium dodecyl sulphate when stirred at a 75 RPM for 60 minutes. Preferably, the composition is administered once daily.

Another embodiment of the composition of the invention comprises 1 part of fenofibrate and 2 parts of organic acid by weight, wherein the organic acid is selected from the group comprising malic acid, tartaric acid, maleic acid, succinic acid and citric acid, and one or more pharmaceutically acceptable excipients. Preferably in this composition, the amount of fenofibrate is 120 mg per dosage which exhibits a dissolution of 54.3 mg of fenofibrate in 1000 mL of neutral pure water containing 2.5 g of sodium dodecyl sulphate when stirred at 75 RPM for 60 minutes. Preferably, the composition is administered once daily.

Another embodiment of the composition of the invention comprises 1 part of fenofibrate and 3 parts of organic acid by weight, wherein the organic acid is selected from the group comprising malic acid, tartaric acid, maleic acid, succinic acid and citric acid, and one or more pharmaceutically acceptable excipients. Preferably in this composition, the amount of fenofibrate is 90 mg per dosage which exhibits a dissolution of 52.9 mg of fenofibrate in 1000 mL of neutral pure water containing 2.5 g of sodium dodecyl sulphate when stirred at 75 RPM for 120 minutes. Preferably, the composition is administered once daily.

Another embodiment of the composition of the invention comprises 1 part of fenofibrate and 5 parts of organic acid by weight, wherein the organic acid is selected from the group comprising malic acid, tartaric acid, maleic acid, succinic acid and citric acid, and one or more pharmaceutically acceptable excipients. Preferably in this composition, the amount of fenofibrate is 90 mg per dosage which exhibits a dissolution of about 54.6 mg of fenofibrate in 1000 mL of neutral pure water containing 2.5 g of sodium dodecyl sulphate when stirred at 75 RPM for 120 minutes. Preferably, the composition is administered once daily.

Another embodiment of the composition of the invention comprises 1 part of fenofibrate and 6 parts of organic acid by weight, wherein the organic acid is selected from the group comprising malic acid, tartaric acid, maleic acid, succinic acid and citric acid, and one or more pharmaceutically acceptable excipients. Preferably in this composition, the amount of fenofibrate is 80 mg per dosage which exhibits a dissolution of about 55 mg of fenofibrate in 1000 mL of neutral pure water containing 2.5 g of sodium dodecyl sulphate when stirred at 75 RPM for 120 minutes. Preferably, the composition is administered once daily.

The amount of dissolution of fenofibrate of LIPANTHYL™ 200 mg capsules is about 55.4 mg per dosage in 1000 mL of neutral pure water containing 2.5 g of sodium dodecyl sulphate when stirred at 75 RPM for 120 minutes. Compared with the LIPANTHYL™ 200 mg capsules, the dissolution amount of the fenofibrate composition of the invention containing different proportions of organic acid and amount of fenofibrate is almost the same as the dissolution amount of the LIPANTHYL™ 200 mg capsules. This means the reduced dose of the fenofibrate composition of the invention containing different amounts of organic acid is bioequivalent with LIPANTHYL™ 200 mg capsules in vitro.

In one embodiment, the method of producing the fenofibrate composition comprises the steps of:
(a) an amount of fenofibrate in the range of 70 mg to 160 mg is dissolved in an organic solvent selected from a group consisting of a ketone such as acetone, butanone and pentanone, dichloromethane, trichloromethane, and an alkanol such as ethanol and isopropyl alcohol, or a mixture thereof;
(b) a water soluble organic acid which is selected from the group comprising malic acid, maleic acid, succinic acid, tartaric acid, fumaric acid and citric acid, are dissolved in the fenofibrate solution of step (a), wherein the amount of organic acid is about 1 to 6 times the weight of the fenofibrate;
(c) the fenofibrate solution of step (a) is sprayed into a fluid bed or granulator containing an expander and filler to form granules with povidone PVPK30, wherein the granules formed are dried in the fluid bed or an oven;
(d) the granules of step (c) are compressed into a tablet or filled into a capsules.

In one embodiment, the method of producing the fenofibrate composition comprises the steps of:
(a) an amount of fenofibrate in the range of 70 mg to 160 mg is suspended in water and the suspension is ground 5 to 10 times;
(b) an organic acid which is selected from the group comprising malic acid, maleic acid, succinic acid, tartaric acid, ascorbic acid and citric acid, are dissolved in the fenofibrate solution or suspension of step (a), wherein the amount of organic acid is about 1 to 6 times the weight of the fenofibrate;
(c) the fenofibrate organic acid solution or mixture of step (b) is sprayed into a fluid bed or granulator containing an expander and filler;
(d) the mixture of step (c) is mixed with povidone (PVPK30) and dried to form granules in the fluid bed or an oven; and
(e) the granules of step (d) comprising the fenofibrate and organic acid are filled into a capsule or compressed into a tablet.

In one embodiment, the fenofibrate composition of the invention is in the form of granules, tablets or capsules.

In one embodiment, the fenofibrate composition of the invention comprises a therapeutically effective amount of fenofibrate and a water soluble organic acid which is selected from the group comprising malic acid, maleic acid, succinic acid, tartaric acid and citric acid, and the amount of the organic acid is from 1 to 6 times the weight of fenofibrate in the composition.

In one embodiment, the fenofibrate composition of the invention comprises 1 part of fenofibrate and 1 to 6 parts of an organic acid selected from the group comprising malic acid, tartaric acid, maleic acid, succinic acid, ascorbic acid and citric acid.

In one embodiment, the fenofibrate composition of the invention comprises 1 part of fenofibrate and 1 to 6 parts of an organic acid which is selected from the group comprising malic acid, tartaric acid, maleic acid, succinic acid and citric acid; wherein the composition further comprises cross-linked povidone, cross-linked carboxymethyl sodium cellulose, a small amount of povidone and sodium dodecyl sulfate.

In one embodiment, a reduced dose composition of fenofibrate comprises fenofibrate, a water soluble organic acid which is selected from the group comprising malic acid, maleic acid, succinic acid, tartaric acid, ascorbic acid and citric acid, wherein the composition further comprises cross-linked povidone and cross-linked carboxymethyl sodium cellulose, and wherein the composition is in the form of granules, a capsule, or a tablet.

In one embodiment, a reduced dose composition of fenofibrate comprises fenofibrate, an organic acid which is selected from the group comprising malic acid, maleic acid, succinic acid, tartaric acid and citric acid, wherein the composition further comprises a cross-linked povidone and cross-linked carboxymethyl sodium cellulose, and wherein the composition is in the form of granules, a capsule, or a tablet.

In one embodiment, the reduced dose composition of fenofibrate comprises 1 part of fenofibrate, 1 to 6 parts of an organic acid selected from the group comprising malic acid, maleic acid, succinic acid, tartaric acid and citric acid.

In one embodiment, the reduced dose composition of fenofibrate comprises 1 part of fenofibrate, 1 to 6 parts of malic acid, cross-linked povidone and cross-linked carboxymethyl sodium cellulose, and wherein the composition is in the form of granules, a capsule, or a tablet.

In one embodiment, the reduced dose composition of fenofibrate comprises 1 part of fenofibrate, 1 to 6 parts of tartaric acid, cross-linked povidone and cross-linked carboxymethyl sodium cellulose, wherein the composition is in the form of granules, a capsule, or a tablet.

In one embodiment, the reduced dose composition of fenofibrate comprises 1 part of fenofibrate, 1 to 6 parts of maleic acid, cross-linked povidone and cross-linked carboxymethyl sodium cellulose, wherein the composition is in the form of granules, a capsule, or a tablet.

In one embodiment, the reduced dose composition of fenofibrate comprises 1 part of fenofibrate, 1 to 6 parts of succinic acid, cross-linked povidone and cross-linked carboxymethyl sodium cellulose, wherein the composition is in the form of granules, a capsule, or a tablet.

In one embodiment, the reduced dose composition of fenofibrate comprises 1 part of fenofibrate, 1 to 6 parts of citric acid, cross-linked povidone and cross-linked carboxymethyl sodium cellulose, wherein the composition is in the form of granules, a capsule, or a tablet.

In one embodiment, the reduced dose composition of fenofibrate comprises fenofibrate, cross-linked povidone, cross-linked carboxymethyl sodium cellulose and maleic acid.

In one embodiment, the reduced dose composition of fenofibrate comprises fenofibrate, cross-linked povidone, cross-linked carboxymethyl sodium cellulose and citric acid.

In one embodiment, the reduced dose composition of fenofibrate comprises fenofibrate, cross-linked povidone, cross-linked carboxymethyl sodium cellulose and succinic acid.

In one embodiment, the reduced dose composition of fenofibrate comprises fenofibrate, cross-linked povidone, cross-linked carboxymethyl sodium cellulose and tartaric acid.

In one embodiment, the reduced dose composition of fenofibrate comprises fenofibrate, cross-linked povidone, cross-linked carboxymethyl sodium cellulose and malic acid.

In one embodiment, the reduced dose composition of fenofibrate comprises fenofibrate, HPMC, an organic acid selected from malic acid, maleic acid, succinic acid, tartaric acid and citric acid, cross-linked povidone, and cross-linked carboxymethyl sodium cellulose.

In one embodiment, the reduced dose composition of fenofibrate comprises fenofibrate, HPC, an organic acid selected from malic acid, maleic acid, succinic acid, tartaric acid and citric acid, cross-linked povidone, and cross-linked carboxymethyl sodium cellulose.

In one embodiment, a reduced dose composition of fenofibrate comprises fenofibrate, an organic acid which is selected from the group comprising malic acid, maleic acid, succinic acid, tartaric acid and citric acid, a carbonate salt selected from calcium carbonate, potassium bicarbonate and sodium bicarbonate, wherein the composition further comprises a cross-linked povidone and cross-linked carboxymethyl sodium cellulose, and wherein the composition is in the form of granules, or a dispersible tablet.

In one embodiment, a reduced dose composition of fenofibrate comprises fenofibrate, an organic acid which is selected from the group comprising malic acid, maleic acid, succinic acid, tartaric acid and citric acid, and calcium carbonate, wherein the composition further comprises a cross-linked povidone and cross-linked carboxymethyl sodium cellulose, and wherein the composition is in the form of granules or a dispersible tablet.

In one embodiment, a reduced dose composition of fenofibrate comprises fenofibrate, an organic acid which is selected from the group comprising malic acid, maleic acid, succinic acid, tartaric acid and citric acid, and sodium bicarbonate, wherein the composition further comprises a cross-linked povidone and cross-linked carboxymethyl sodium cellulose, and wherein the composition is in the form of granules or a dispersible tablet.

### BRIEF DESCRIPTION OF THE FIGURES

FIG. 1 represents the comparative plasma level of fenofibric acid of a LIPANTHYL™ 160 mg tablet and a fenofibrate composition of the invention under fasting condition.

FIG. 2 represents the comparative plasma level of fenofibric acid of a LIPANTHYL™ 160 mg tablet and a fenofibrate composition of the invention underfed condition.

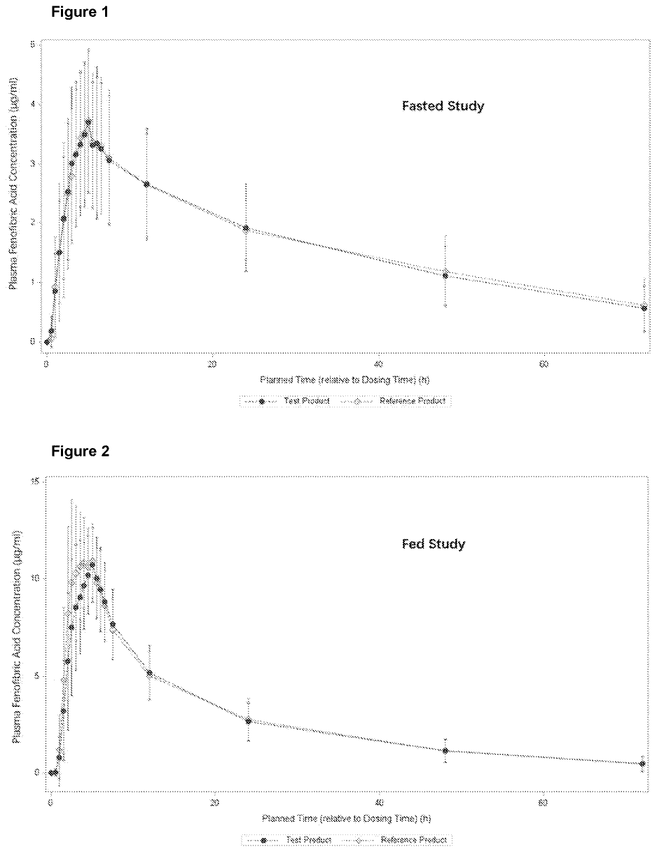

### DETAILED DESCRIPTION OF THE INVENTION

The present invention provides discloses a reduced dose composition of fenofibrate containing 0 to 6 parts of organic acid. The specification further discloses a reduced dose composition comprising 1 parts of fenofibrate, 0 to 6 parts of an organic acid selected from a group comprising malic acid, tartaric acid, maleic acid, succinic acid, ascorbic acid, fumaric acid and citric acid, and one or more pharmaceutically acceptable excipients selected from cross-linked povidone, cross-linked carboxymethyl sodium cellulose, lactose, sucrose, HPMC and HPC, the composition having improved dissolution properties in an aqueous medium of 1000 mL neutral water containing 2.5 g of sodium dodecyl sulphate.

The composition of the invention containing 160 mg of fenofibrate with no organic acid exhibits substantial bioequivalence to LIPANTHYL™ 160 mg under fasted and fed conditions. The 160 mg fenofibrate tablet of the invention is made by mixing fenofibrate acetone solution with cross-linked povidone, cross-linked carboxymethyl sodium cellulose and lactose in a fluid bed and contains no organic acid. The mixture of fenofibrate, cross-linked povidone, cross-linked carboxymethyl sodium cellulose and lactose is dried in the fluid bed and then compressed into a tablet after mixing with magnesium stearate.

LIPANTHYL™ 160 mg comprises micronized fenofibrate made by mechanical force, whereas the 160 mg fenofibrate tablet of the invention is made by using acetone to dissolve the ordinary fenofibrate raw material to produce the composition of the invention. The fenofibrate can be dissolved by other organic solvents such as butanone, pentanone, dichloromethane, trichloromethane, and alkanols such as ethanol and isopropyl alcohol, or mixtures thereof. The composition of the invention made using acetone solution makes it effective at lower doses as well as improving the high dose-associated side effect profile of fenofibrate.

The composition also offers a method of treatment of primary hyperlipidemias, hypercholesterolemia or hypertriglyceridemia comprising administering a reduced dose oral pharmaceutical composition of fenofibrate to a patient with or without food.

The invention provides a process of manufacturing reduced dose compositions comprising a mixture of fenofibrate and 1 to 6 times the amount of organic acid, wherein the organic acid is a water soluble excipient or chemical selected from a group comprising malic acid, tartaric acid, maleic acid, succinic acid, ascorbic acid and citric acid, and one or more pharmaceutically acceptable excipients selected from cross-linked povidone, cross-linked carboxymethyl sodium cellulose and lactose.

Fenofibrate includes amorphous or crystalline polymorphic forms of fenofibrate, and mixtures thereof. The term “mixture” refers generally to a system having one or more components which are mixed but not combined chemically.

The USFDA has approved fenofibrate tablets as well as capsules which contain different doses of fenofibrate. The Lipidil® capsule of Abbott contains 100 mg of fenofibrate to be administered 3 times daily. The Tricor® Micronised capsule of Abbott contains 67, 134 or 200 mg of fenofibrate. The Tricor® tablet of Abbott contains 54 or 160 mg of fenofibrate. The prescription dosage form of fenofibrate such as the Tricor® tablet of Abbott contains 48 or 145 mg, the Lipophen® capsule of Kowa Pharmaceuticals contains 50, 100 or 150 mg, the Fenoglide® tablet of SALIX Pharma contains 40 or 120 mg, the Antara® capsule of Lupin Atlantis contains 43 or 130 mg of fenofibrate.

The term “reduced dose”, as used herein, refers to a dose fenofibrate that is less than 200 mg but is bioequivalent with LIPANTHYL™ 200 mg capsules in vitro. LIPANTHYL™ 200 mg capsules contain 200 mg of micronised fenofibrate, lactose, magnesium stearate, pregelatinised maize starch, sodium laurilsulfate, crospovidone, titanium dioxide (E171), iron oxide (E172) erythrosine (E127) and gelatin.

In one embodiment, the fenofibrate in the compositions provided herein is not micronized.

Advantages of the compositions described herein include, but are not limited to: lower doses to obtain the same pharmacological effect as a conventional dose; and increased bioavailability.

In one embodiment, the present invention provides a method of producing a fenofibrate tablet by using fenofibrate in an organic solvent selected from a group comprising a ketone such as acetone, butanone and pentanone, dichloromethane, trichloromethane, and an alkanol such as ethanol and isopropyl alcohol, or a mixture thereof; wherein the fenofibrate acetone solution is sprayed into a fluid bed containing the disintegrating materials selected from cross-linked carboxymethyl sodium cellulose, cross povidone, lactose and microcrystalline cellulose, and followed by addition of aqueous PVPK30 as the binder. Preferably, the granules formed are compressed into a tablet or filled into a capsule.

The term “dissolution rate”, in relation to formulations including tablets and capsules, is used by the US Pharmacopoeia (USP) to refer to the percentage of active pharmaceutical ingredient (API) of the formulation detected under specific conditions from stirring at 50 to 100 rotations per minute, and for 30 to 120 minutes at 37° C. in a USP dissolution machine. The dissolution medium may have a different composition, a different pH and/or a different rotations per minute rate.

For fenofibrate, the dissolution medium of USP is pH 7 water containing 0.72% sodium dodecyl sulfate (SDS) and the specification is over 85% within 45 minutes. The high content of SDS increases the dissolution of fenofibrate in neutral water and cannot distinguish different dissolution rates of different formulations of fenofibrate. Therefore, a dissolution medium of 0.25% SDS is preferred to differentiate the dissolution rate of different formulations in the present invention.

In one embodiment, the composition of each dosage comprises 160 mg fenofibrate, cross-linked povidone, crosslinked carboxymethyl sodium cellulose, a small amount of PVPK30 and a lubricating agent magnesium stearate, or a mixture thereof. The composition may be in the form of granules, a capsule or a tablet. Preferably, the composition is bioequivalent in vitro and in vivo with a LIPANTHYL™ 160 mg fenofibrate tablet.

In another embodiment, the composition comprises both fenofibrate and a water soluble organic acid selected from the group comprising malic acid, maleic acid, succinic acid, tartaric acid, ascorbic acid and citric acid, or a mixture thereof. The composition may further comprise cross-linked povidone, crosslinked carboxymethyl sodium cellulose, a small amount of PVPK30 and a lubricating agent magnesium stearate, or a mixture thereof. The composition may be in the form of a tablet, granules or a capsule.

In another embodiment, the invention comprises 145 mg or 1 part of fenofibrate, 145 mg or 1 part of a water soluble organic acid selected from the group comprising malic acid, maleic acid, succinic acid, tartaric acid and citric acid, or a mixture thereof, 100 mg to 150 mg cross-linked povidone, 100 mg to 150 mg crosslinked carboxymethyl sodium cellulose, a small amount of PVPK30, sodium dodecyl sulfate and magnesium stearate. Preferably, the dissolution amount of the 145 mg fenofibrate tablet of the invention is about the same amount as a LIPANTHYL™ 200 mg fenofibrate capsule. Preferably, the composition is bioequivalent in vitro with a LIPANTHYL™ 200 mg fenofibrate capsule.

In another embodiment, the composition comprises different proportions of water soluble organic acid relative to fenofibrate. The water soluble organic acid may be selected from the group selected from malic acid, maleic acid, succinic acid, tartaric acid and citric acid, or a mixture thereof. Preferably, the composition further comprises cross-linked povidone, crosslinked carboxymethyl sodium cellulose, a small amount of PVPK30, SDS and magnesium stearate, or a mixture thereof. Preferably, the composition is bioequivalent in vitro with a LIPANTHYL™ 200 mg fenofibrate capsule. The composition may be in the form of a tablet, granules or a capsule.

In another embodiment, the invention comprises 120 mg fenofibrate, 240 mg of a water soluble organic acid selected from the group comprising malic acid, maleic acid, succinic acid, tartaric acid and citric acid, or a mixture thereof, 100 mg to 150 mg cross-linked povidone, 100 mg to 150 mg cross-linked carboxymethyl sodium cellulose, a small amount of PVPK30, sodium dodecyl sulfate and magnesium stearate. The dissolution amount of the 120 mg fenofibrate tablet or granules of the invention is about 54.5 mg and is about the same amount as released by LIPANTHYL™ 200 mg fenofibrate capsules when stirred in 1000 mL of neutral pure water containing 2.5 g of sodium dodecyl sulphate at a blade rotation speed of 75 RPM for 120 minutes.

In another embodiment, the invention comprises 90 mg of fenofibrate, 270 mg of a water soluble organic acid selected from the group comprising malic acid, maleic acid, succinic acid, tartaric acid and citric acid, or a mixture thereof, 70 mg to 100 mg cross-linked povidone, 70 mg to 100 mg cross-linked carboxymethyl sodium cellulose, a small amount of PVPK30, sodium dodecyl sulfate and magnesium stearate. The dissolution amount of the 90 mg fenofibrate tablet or granules of the invention is about 52.6 mg and is similar to the dissolution amount of LIPANTHYL™ 200 mg fenofibrate capsules when stirred in 1000 mL of neutral pure water containing 2.5 g of sodium dodecyl sulphate at a blade rotation speed of 75 RPM for 120 minutes.

In another embodiment, the invention comprises 90 mg fenofibrate, 450 mg of a water soluble organic acid selected from the group comprising malic acid, maleic acid, succinic acid, tartaric acid and citric acid, or a mixture thereof, 75 mg to 150 mg of cross-linked povidone, 75 mg to 150 mg of cross-linked carboxymethyl sodium cellulose, a small amount of PVPK30, sodium dodecyl sulfate and magnesium stearate. The dissolution amount of the 90 mg fenofibrate tablet or granules of the invention is about 54.6 mg and is about the same dilution amount as LIPANTHYL™ 200 mg fenofibrate capsules when stirred in 1000 mL of neutral pure water containing 2.5 g of sodium dodecyl sulphate at a blade rotation speed of 75 RPM for 120 minutes.

In another embodiment, the invention comprises 80 mg fenofibrate, 480 mg of a water soluble organic acid selected from the group comprising malic acid, maleic acid, succinic acid, tartaric acid and citric acid, or a mixture thereof, 80 mg to 150 mg cross-linked povidone, 80 mg to 150 mg cross-linked carboxymethyl sodium cellulose, a small amount of PVPK30, sodium dodecyl sulfate and magnesium stearate. The dissolution amount of the 80 mg fenofibrate tablet or granules of the invention is about 56 mg and is about the same amount as the dilution of LIPANTHYL™ 200 mg fenofibrate capsules when stirred in 1000 mL of neutral pure water containing 2.5 g of sodium dodecyl sulphate at a blade rotation speed of 75 RPM for 120 minutes.

In one embodiment, the ratio of the weight of water soluble organic acid to fenofibrate is 1 to 1 by weight. In another embodiment, the ratio of the weight of water soluble organic acid to fenofibrate is 2 to 1 by weight. In another embodiment, the ratio of the weight of water soluble organic acid to fenofibrate is 3 to 1 by weight. In another embodiment, the ratio of the weight of water soluble organic acid to fenofibrate is 4 to 1 by weight. In another embodiment, the ratio of the weight of water soluble organic acid to fenofibrate is 5 to 1 by weight. In another embodiment, the ratio of the weight of water soluble organic acid to fenofibrate is 6 to 1 by weight. In preferred embodiments, the ratio of the weight of water soluble organic acid to fenofibrate in the composition is from 1:1 to 6:1 by weight.

In one embodiment, the composition comprises fenofibrate, cross-linked povidone, cross-linked sodium carboxymethyl cellulose, a small amount of sodium dodecyl sulfate, and magnesium stearate, wherein the composition is in the form of a tablet, a capsule or granules.

In one embodiment, the method of producing a fenofibrate composition comprises the following steps:
a) 1 part of fenofibrate is dissolved in 1 to 4 parts of an organic solvent, wherein the solvent may be selected from acetone, butanone, pentanone, dichloromethane, trichloromethane, ethanol or isopropyl alcohol
b) the solution formed in step a) is sprayed into a fluid bed containing 1 to 3 parts of cross-linked povidone, 1 to 3 parts of cross-linked sodium carboxymethyl cellulose, sugar and a small amount of sodium dodecyl sulfate (25-50 mg per tablet, capsule or granules bag);
c) an 8% PVPK30 aqueous solution is spayed into the fluid bed as a binder to form granules;
d) after the size of the granules produced in step c) reaches about 24 mesh and the water content is below 4%, the granulation is stopped and the granules are mixed with magnesium stearate; and
e) the granules are compressed into a tablet or filled into a capsule or granules bag.

In another embodiment, the composition comprises 1 part of fenofibrate and 1 to 6 parts by weight of a water soluble organic acid selected from the group comprising malic acid, maleic acid, succinic acid, citric acid and tartaric acid. Preferably, the composition further comprises cross-linked povidone, cross-linked sodium carboxymethyl cellulose, a small amount of sodium dodecyl sulfate and magnesium stearate, and the composition is in the form of a tablet, a capsule or granules. In this embodiment, the method of producing a fenofibrate composition comprising a water soluble organic acid comprises the following steps:
a) one part of fenofibrate and 1 to 6 parts of organic acid is dissolved in about 6 to 18 parts of an organic solvent, wherein the solvent is a ketone, such as acetone, butanone, pentanone, or a mixture thereof, or wherein the solvent is dichloromethane, trichloromethane, ethanol and isopropyl alcohol, or a mixture thereof; wherein the water soluble organic acid is selecting from the group comprising malic acid, maleic acid, succinic acid, citric acid and tartaric acid;
b) the solution formed in step a) is sprayed into a fluid bed containing about 1 part of cross-linked povidone, 1 part of cross-linked sodium carboxymethyl cellulose and a small amount of sodium dodecyl sulfate (25-50 mg per tablet, capsule or granules bag)—the organic solvent is evaporated in the fluid bed and is not contained in the composition of the invention;
c) an 8% PVPK30 aqueous solution is spayed into the fluid bed as the binder to form granules;
d) after the size of granules produced in step c) reaches about 24 mesh and the water content is below 4%, the granulation is stopped and the granules are mixed with magnesium stearate; and
e) the granules are compressed into a tablet or filled into a capsule or granules bag.

Preferably, the composition of the invention has a dissolution rate of about 38% to 68% of fenofibrate after stirring at 75 RPM for 120 minutes in 1000 mL of neutral pure water containing 2.5 g of SDS. The dissolution amount is proportional to the ratio of the organic acid to the fenofibrate in the composition. Preferably, the greater the amount of organic acid in the composition relative to fenofibrate, the higher the dissolution rate of fenofibrate of the composition.

In another embodiment, the method of production of the fenofibrate composition comprises mixing the fenofibrate/acetone solution with the cross-linked carboxymethyl sodium cellulose, cross-linked povidone, SDS, PVPK30 in a fluid bed or wet granulator; and then dried by heat in the machine. The granules are mixed with magnesium stearate, wherein the composition is made in the form of a tablet, a capsule or granules.

In another embodiment, the method of producing a fenofibrate composition comprises the steps of:
a) dissolving an amount of fenofibrate in an organic solvent, wherein the solvent is a ketone selected from acetone, butanone or pentanone, dichloromethane, trichloromethane, ethanol, isopropyl alcohol, or a mixture thereof;
b) mixing the fenofibrate solution of a) with an organic acid selected from the group comprising malic acid, maleic acid, succinic acid, tartaric acid and citric acid;
c) mixing the mixture of b) with one or more excipients selected from a group consisting of cross-linked povidone, and cross-linked carboxymethyl sodium cellulose, in a fluid bed or wet granulator;
d) mixing the mixture of c) with povidone (PVPK30) and dried to form granules in a fluid bed or oven; and
e) compressing the granules formed in step d) into a tablet or filling the granules of step d) into a capsule.

In another embodiment, the method of production of the composition comprises dissolving 1 part of fenofibrate and 1 to 6 parts of malic acid in acetone. The resulting solution is then mixed with 0.8 to 2 parts of cross-linked povidone, 0.8 to 2 parts of cross-linked carboxymethyl sodium cellulose, and a small amount of SDS, in a fluid bed or wetting granulator. The powder formed is mixed with or stuck together with PVPK30 to form granules, which are compressed into a tablet or filled into a capsule. Preferably, the composition comprises fenofibrate, malic acid, cross-linked povidone, cross-linked carboxymethyl sodium cellulose, and a small amount of SDS.

In another embodiment, the method of production of the composition comprises 1 part of fenofibrate, 1 to 6 parts of an organic acid selected from malic acid, maleic acid, tartaric acid, succinic acid and citric acid, 0.8 to 2 parts of cross-linked povidone, 0.8 to 2 parts of cross-linked carboxymethyl sodium cellulose, a small amount of SDS, and a carbonate salt selected from calcium carbonate, sodium bicarbonate and potassium bicarbonate, such that the formulation is in the form of a dispersible tablet.

In another embodiment, the method of production of the composition comprises 1 part of fenofibrate, 1 to 6 parts of malic acid, 0.8 to 2 parts of cross-linked povidone, 0.8 to 2 parts of cross-linked carboxymethyl sodium cellulose, a small amount of SDS, and a carbonate salt selected from calcium carbonate, sodium bicarbonate and potassium bicarbonate, such that the formulation is in the form of a dispersible tablet.

In another embodiment, the method of production of the composition comprises 1 part of fenofibrate, 1 to 6 parts of maleic acid, 0.8 to 2 parts of cross-linked povidone, 0.8 to 2 parts of cross-linked carboxymethyl sodium cellulose, a small amount of SDS, and a carbonate salt selected from calcium carbonate, sodium bicarbonate and potassium bicarbonate, such that the formulation is in the form of a dispersible tablet.

In another embodiment, the method of production of the composition comprises 1 part of fenofibrate, 1 to 6 parts of tartaric acid, 0.8 to 2 parts of cross-linked povidone, 0.8 to 2 parts of cross-linked carboxymethyl sodium cellulose, a small amount of SDS, and a carbonate salt selected from calcium carbonate, sodium bicarbonate and potassium bicarbonate, such that the formulation is in the form of a dispersible tablet.

In another embodiment, the method of production of the composition comprises 1 part of fenofibrate, 1 to 6 parts of citric acid, 0.8 to 2 parts of cross-linked povidone, 0.8 to 2 parts of cross-linked carboxymethyl sodium cellulose, a small amount of SDS, and a carbonate salt selected from calcium carbonate, sodium bicarbonate and potassium bicarbonate, such that the formulation is in the form of a dispersible tablet.

In another embodiment, the method of production of the composition comprises 1 part of fenofibrate, 1 to 6 parts of succinic acid, 0.8 to 2 parts of cross-linked povidone, 0.8 to 2 parts of cross-linked carboxymethyl sodium cellulose, a small amount of SDS, and a carbonate salt selected from calcium carbonate, sodium bicarbonate and potassium bicarbonate, such that the formulation is in the form of a dispersible tablet.

In another embodiment, the method of production of the composition comprises dissolving 1 part of fenofibrate and 1 to 6 parts of maleic acid in acetone. The resulting solution is then mixed with 0.8 to 2 parts of cross-linked povidone, 0.8 to 2 parts of cross-linked carboxymethyl sodium cellulose, and a small amount of SDS, in a fluid bed or wetting granulator. The powder formed is mixed with or stuck together with PVPK30 to form granules, which are compressed into a tablet or filled into a capsule. Preferably, the composition comprises fenofibrate, maleic acid, cross-linked povidone, cross-linked carboxymethyl sodium cellulose, and a small amount of SDS.

In another embodiment, the method of production of the composition comprises dissolving 1 part of fenofibrate and 1 to 6 parts of succinic acid into acetone. The resulting solution is then mixed with 0.8 to 2 parts of cross-linked povidone, 0.8 to 2 parts of cross-linked carboxymethyl sodium cellulose, and a small amount of SDS, in a fluid bed or wetting granulator. The powder formed is mixed with or stuck together with PVPK30 to form granules, which are compressed into a tablet or filled into a capsule. Preferably, the composition comprises fenofibrate, succinic acid, cross-linked povidone, cross-linked carboxymethyl sodium cellulose, and a small amount of SDS.

In another embodiment, the method of production of the composition comprises dissolving 1 part of fenofibrate and 1 to 6 parts of tartaric acid in acetone. The resulting solution is then mixed with 0.8 to 2 parts of cross-linked povidone, 0.8 to 2 parts of cross-linked carboxymethyl sodium cellulose, and a small amount of SDS, in a fluid bed or wetting granulator. The powder formed is mixed with or stuck together with PVPK30 to form granules, which are compressed into a tablet or filled into a capsule. Preferably, the composition comprises fenofibrate, tartaric acid, cross-linked povidone, cross-linked carboxymethyl sodium cellulose, and a small amount of SDS.

In another embodiment, the method of production of the composition comprises dissolving 1 part of fenofibrate and 1 to 6 parts of citric acid in acetone. The resulting solution is then mixed with 0.8 to 2 parts of cross-linked povidone, 0.8 to 2 parts of cross-linked carboxymethyl sodium cellulose, and a small amount of SDS, in a fluid bed or wetting granulator. The powder formed is mixed with or stuck together with PVPK30 to form granules, which are dried in the fluid bed or an oven, and which are then compressed into a tablet or filled into a capsule. Preferably, the composition comprises fenofibrate, citric acid, cross-linked povidone, cross-linked carboxymethyl sodium cellulose, and a small amount of SDS.

In another embodiment, the composition of the invention comprising 90 mg of fenofibrate and 450 mg of citric acid and has a dissolution amount of about 56.4 mg of fenofibrate after stirring at 75 RPM for 120 minutes in 1000 mL of neutral water containing 2.5 g of sodium dodecyl sulfate, which is almost the same as the dissolution amount of fenofibrate from a LIPANTHYL™ 200 mg fenofibrate capsule under the same dissolution conditions. This means 1 tablet of the 90 mg fenofibrate composition of the invention may be bio-equivalent to a 200 mg LIPANTHYL™ capsule of the original producer in vitro.

In some embodiments, the ratio of water soluble organic acid to fenofibrate is in a range from 1:1 to 6:1 by weight. Preferably, the water soluble organic acid is selected from the group comprising malic acid, maleic acid, succinic acid, tartaric acid and citric acid, or a mixture thereof.

In some embodiments of the fenofibrate composition, there is no organic acid, and the composition comprises an expander such as cross-linked povidone, cross-linked sodium carboxymethyl cellulose, lactose, sucrose, a small amount of sodium dodecyl sulfate, povidone and magnesium stearate.

In another embodiment, the fenofibrate composition comprises a water soluble organic acid, wherein the ratio of organic acid to fenofibrate is 1:1 by weight, wherein the water soluble organic acid is selected from the group comprising malic acid, maleic acid, succinic acid, tartaric acid and citric acid, and the fenofibrate composition further comprises an expander such as cross-linked povidone, cross-linked sodium carboxymethyl cellulose, lactose, sucrose, a small amount of sodium dodecyl sulfate, povidone and magnesium stearate.

In another embodiment, the fenofibrate composition comprises a water soluble organic acid, wherein the ratio of water soluble organic acid to fenofibrate is 2:1 by weight, wherein the organic acid is selected from the group comprising malic acid, maleic acid, succinic acid, tartaric acid and citric acid, and the fenofibrate composition further comprises an expander such as cross-linked povidone, cross-linked sodium carboxymethyl cellulose, lactose, sucrose, a small amount of sodium dodecyl sulfate, povidone and magnesium stearate.

In another embodiment, the fenofibrate composition comprises a water soluble organic acid, wherein the ratio of water soluble organic acid to fenofibrate is 3:1 by weight, wherein the water soluble organic acid is selected from the group comprising malic acid, maleic acid, succinic acid, tartaric acid, and citric acid, and the fenofibrate composition further comprises an expander such as cross-linked povidone, cross-linked sodium carboxymethyl cellulose, lactose, sucrose, a small amount of sodium dodecyl sulfate, povidone and magnesium stearate.

In another embodiment, the fenofibrate composition comprises a water soluble organic acid, wherein the ratio of water soluble organic acid to fenofibrate is 4:1 by weight, wherein the water soluble organic acid is selected from the group comprising malic acid, maleic acid, succinic acid, tartaric acid and citric acid, and the fenofibrate composition further comprises an expander such as cross-linked povidone, cross-linked sodium carboxymethyl cellulose, lactose, sucrose, a small amount of sodium dodecyl sulfate, povidone and magnesium stearate.

In another embodiment, the fenofibrate composition comprises a water soluble organic acid, wherein the ratio of water soluble organic acid to fenofibrate is 5:1 by weight, wherein the water soluble organic acid is selected from the group comprising malic acid, maleic acid, succinic acid, tartaric acid and citric acid, and the fenofibrate composition further comprises an expander such as cross-linked povidone, cross-linked sodium carboxymethyl cellulose, lactose, sucrose, sodium dodecyl sulfate, a small amount of povidone and magnesium stearate.

In another embodiment, the fenofibrate composition comprises a water soluble organic acid, wherein the ratio of water soluble organic acid to fenofibrate is 6:1 by weight, wherein the water soluble organic acid is selected from the group comprising malic acid, maleic acid, succinic acid, tartaric acid and citric acid, and the fenofibrate composition further comprises an expander such as cross-linked povidone, cross-linked sodium carboxymethyl cellulose, lactose, sucrose, sodium dodecyl sulfate, a small amount of povidone and magnesium stearate.

In one embodiment, the fenofibrate composition of the invention may be produced by dissolving 1 part of fenofibrate in 2 to 3 parts of acetone. Then the fenofibrate acetone solution is sprayed into a fluid bed comprising the excipients lactose, sucrose, microcrystalline cellulose, cross-linked povidone, cross-linked carboxymethyl sodium cellulose, and a small amount of a sodium dodecyl sulfate. The resultant powder obtained is mixed with or stuck together with povidone or PVPK30 to form granules, which are dried in the fluid bed or an oven. The granules are mixed with magnesium stearate and then compressed into a tablet, filled into a capsule or filled into a granules bag. Preferably, the resultant composition has higher dissolution rate in aqueous medium and is in the form of granules, a tablet or a capsule for the treatment of hyperlipidemia. Preferably, this composition has a dissolution rate of fenofibrate of about 35% to 60% when stirred at 75 RPM (rotations per minute) at 120 minutes in 1000 mL of neutral pure water containing 2.5 g of sodium dodecyl sulfate.

In another embodiment, the invention may comprise fenofibrate and a water soluble organic acid. Preferably, the ratio of the weight of organic acid to fenofibrate is in the range of 1:1 to 6:1. Preferably, the composition has higher dissolution rate in aqueous medium and is in the form of granules, a tablet or a capsule for the treatment of hyperlipidemia. Preferably, the water soluble organic acid of the composition of fenofibrate is selected from the group comprising malic acid, maleic acid, succinic acid, tartaric acid and citric acid.

In some embodiments, the fenofibrate composition comprises fenofibrate, a water soluble organic acid selected from the group comprising tartaric acid, maleic acid, succinic acid, malic acid and citric acid, cross-linked povidone, cross-linked carboxymethyl sodium cellulose, a small amount of povidone, 20-50 mg of SDS in each composition, and magnesium stearate, wherein the composition is in the form of granules, a capsule or a tablet.

In another embodiment, the invention comprises fenofibrate, a water soluble organic acid selected from the group comprising tartaric acid, maleic acid, succinic acid, malic acid and citric acid, and carbonate salt selected from calcium carbonate, sodium bicarbonate and potassium bicarbonate. Preferably, the composition has high dissolution rate and fast disintegration in aqueous medium and is in the form of granules or dispersible tablet for the treatment of hyperlipidemia.

In some embodiments, the fenofibrate composition comprises fenofibrate, a water soluble organic acid selected from the group comprising tartaric acid, maleic acid, succinic acid, malic acid, and citric acid, cross-linked povidone, cross-linked carboxymethyl sodium cellulose, a small amount of povidone, and magnesium stearate, wherein the composition has a dissolution rate of fenofibrate of more than 38% or in the range of 38% to 68% when stirred at 75 RPM (rotations per minute) at 120 minutes in 1000 mL of neutral water containing 2.5 g of sodium dodecyl sulfate. Preferably, a greater amount of organic acid used corresponds to a higher fenofibrate dissolution rate.

In some embodiments, the amount of water soluble organic acid is from 1 to 6 times the weight of fenofibrate in the composition. Preferably, the composition comprises cross-linked carboxymethyl sodium cellulose, cross-linked povidone, lactose, sucrose, 2-5% of povidone as the binder, and magnesium stearate, wherein the composition is in the form of granules, a capsule or a tablet.

In another embodiment, the fenofibrate composition comprises fenofibrate, malic acid, cross-linked povidone, cross-linked carboxymethyl sodium cellulose, lactose, sucrose, 2-5% of povidone as the binder, and magnesium stearate, wherein the composition is in the form of granules, a capsule or a tablet, and wherein the composition has a fenofibrate dissolution rate of 38% (55 mg/145 mg) to 68% (55 mg/80 mg) when stirred at 75 RPM for 120 minutes in 1000 mL of neutral water containing 2.5 g of sodium dodecyl sulfate. Preferably, the ratio of malic acid to fenofibrate ranges from 1:1 to 6:1 by weight/weight ratio in the composition.

In another embodiment, the fenofibrate composition comprises fenofibrate, maleic acid, cross-linked povidone, cross-linked carboxymethyl sodium cellulose, lactose, sucrose, 2-5% of povidone as the binder, and magnesium stearate, wherein the composition is in the form of granules, a capsule or a tablet, and wherein the composition has a fenofibrate dissolution rate of 38% to 68% when stirred at 75 RPM for 120 minutes in 1000 mL of neutral water containing 2.5 g of sodium dodecyl sulfate. Preferably, the ratio of maleic acid to fenofibrate ranges from 1:1 to 6:1 by weight/weight in the composition.

In another embodiment, the fenofibrate composition comprises fenofibrate, tartaric acid, cross-linked povidone, cross-linked carboxymethyl sodium cellulose, lactose, sucrose, 2-5% of povidone as the binder, and magnesium stearate, wherein the composition is in the form of granules, capsule or tablet, and wherein the composition has a fenofibrate dissolution rate of 38% to 68% when stirred at 75 RPM for 120 minutes in 1000 mL of neutral water containing 2.5 g of sodium dodecyl sulfate. Preferably, the ratio of tartaric acid to fenofibrate ranges from 1:1 to 6:1 by weight/weight in the composition.

In another embodiment, the fenofibrate composition comprises fenofibrate, citric acid, cross-linked povidone, cross-linked carboxymethyl sodium cellulose, lactose, sucrose, 2-5% of povidone as the binder and magnesium stearate, wherein the composition is in the form of granules, capsule or tablet and has a dissolution rate of 38% to 68% of fenofibrate when stirred at 75 RPM for 120 minutes in 1000 mL of neutral water containing 2.5 g of sodium dodecyl sulfate. Preferably, the ratio of citric acid to fenofibrate ranges from 1:1 to 6:1 by weight/weight in the composition.

In another embodiment, the fenofibrate composition comprises fenofibrate, succinic acid, cross-linked povidone, cross-linked carboxymethyl sodium cellulose, lactose, sucrose, 2-5% of povidone as the binder and magnesium stearate, wherein the formulation is in the form of granules, capsule or tablet, and has a fenofibrate dissolution rate of 38% to 68% when stirred at 75 RPM for 120 minutes in 1000 mL of neutral water containing 2.5 g of sodium dodecyl sulfate. Preferably, the ratio of succinic acid to fenofibrate ranges from 1:1 to 6:1 by weight/weight in the composition.

The present invention broadly relates to a convenient drug delivery system, and methods of producing a drug delivery system, preferably using common water soluble excipients or chemicals via a simple method and at low cost. There is no need to grind the water insoluble fenofibrate in water to form a water suspension and no grinding machine is needed. The organic acid is a food nutrient which is permitted to be consumed in amounts far beyond the quantities required in compositions of the invention. More particularly, the present invention relates to immediate release dosage forms and methods for their production and administration.

The higher dissolution of fenofibrate of the present invention compared with LIPANTHYL™ 200 mg fenofibrate capsules on the market may reduce the amount of fenofibrate needed to treat hyperlipidaemia, which may in turn reduce the alimentary side effects of fenofibrate. Preferably, the ratio of the weight of water soluble organic acid to fenofibrate ranges from 1:1 to 6:1.

The composition of a 160 mg fenofibrate tablet of the invention, as described herein, produced by the spraying of fenofibrate ketone solution onto a mixture of cross-linked povidone and cross-linked sodium carboxymethyl cellulose to form a powder mixture, has the same dissolution rate as the LIPANTHYL™ 200 mg fenofibrate capsule and has a similar dissolution rate as the LIPANTHYL™ 160 mg fenofibrate tablet. Further increasing the dissolution rate of the fenofibrate composition, as described herein, requires the addition of water soluble organic acid into the fenofibrate composition; the fenofibrate organic acid solution is sprayed into a fluid bed containing an expander such as cross-linked povidone and cross-linked sodium carboxymethyl cellulose to form a powder mixture.

Preferably, the greater the amount of water soluble organic acid in the composition, the higher the dissolution rate of fenofibrate in an aqueous medium. In one embodiment, if the amount of fenofibrate in the composition or unit dosage is 145 mg, the ratio of organic acid to fenofibrate is about 1:1. In another embodiment, if the amount of fenofibrate in each composition or unit dosage is 120 mg, the ratio of organic acid to fenofibrate is about 2:1 in order to release 56 mg of fenofibrate, i.e., which is the dissolution amount of a LIPANTHYL™ 200 mg fenofibrate capsule, when stirred at 75 RPM for 120 minutes in 1000 mL of 0.25% sodium dodecyl sulfate water solution.

In another embodiment, if the amount of fenofibrate in each composition or unit dosage is 90 mg, the ratio of water soluble organic acid to fenofibrate is in the range of about 4:1 to 5:1 in order to release 52 mg to 56 mg of fenofibrate when stirred at 75 RPM for 120 minutes in 1000 mL of neutral water containing 2.5 g of sodium dodecyl sulfate.

In another embodiment, if the amount of fenofibrate in each composition or unit dosage is 80 mg, the ratio of water soluble organic acid to fenofibrate is 6:1 in order to release 55 mg of fenofibrate when stirred at 75 RPM for 120 minutes in 1000 mL of neutral water containing 2.5 g of sodium dodecyl sulfate. Preferably, the amount of fenofibrate in each composition is equal to or less than 160 mg. In a preferred embodiment, the amount of fenofibrate in an oral composition is 145 mg per unit dosage or tablet. In another preferred embodiment, the amount of fenofibrate contained in the composition is 120 mg per unit dosage or tablet. In another preferred embodiment, the amount of fenofibrate contained in the composition is 90 mg per unit dosage or tablet. In another preferred embodiment, the amount of fenofibrate in the oral composition is 80 mg per unit dosage. Preferably, such compositions have a rate of fenofibrate dissolution in the range of 35-68% or, preferably, all such compositions have the same amount of fenofibrate dissolution of about 55 mg, when stirred at 75 RPM for 120 minutes in 1000 mL of neutral water containing 2.5 g of sodium dodecyl sulfate.

The amount of dissolution of fenofibrate of a LIPANTHYL™ 200 mg capsules when subjected to the same conditions is about 55 mg, i.e., when stirred at 75 RPM for 120 minutes in 1000 mL of neutral water containing 2.5 g of sodium dodecyl sulfate. This means 1 tablet of compositions of invention comprising 80 mg fenofibrate, 90 mg fenofibrate, 120 mg fenofibrate, or 145 mg fenofibrate with different amounts of organic acid, is bioequivalent with a LIPANTHYL™ 200 mg fenofibrate capsule of the original producer in vitro. The water soluble organic acid in the compositions act as a dispersion agent to raise the dissolution of fenofibrate in the composition which is better than micronization of the raw material by mechanical force.

In a preferred embodiment, the amount of fenofibrate in a composition of the invention is in the range of 80 mg to 160 mg, wherein the composition may be in the form of a tablet, granules or a capsule.

The fenofibrate compositions produced by the methods disclosed herein are useful to reduce total cholesterol, LDL cholesterol, apolipoprotein B, total triglycerides, and triglyceride rich lipoprotein (VLDL) in human subjects.

The reduced dose composition comprises fenofibrate, organic acid, HPMC and/or HPC of low molecular weight, a core comprising sugar and/or microcrystalline cellulose, wherein the composition is in the form of sphere or capsules.

The fenofibrate compositions of the invention can additionally comprise one or more compounds useful in treating dyslipidemia, hyperlipidemia, hypercholesterolemia, cardiovascular disorders, or related conditions, or the fenofibrate compositions can be administered in conjunction with such a compound. Examples of suitable compounds include, but are not limited to, statins or HMG CoA reductase inhibitors and antihypertensives. Examples of antihypertensives include, but are not limited to diuretics, beta blockers, alpha blockers, alpha-beta blockers, sympathetic nerve inhibitors, angiotensin converting enzyme (ACE) inhibitors, calcium channel blockers, and angiotensin receptor blockers.

### Definitions

In order for the invention described herein to be more fully understood, the following definitions are provided for the purposes of this disclosure.

The term “comprising” is an inclusive term interpreted to mean containing, embracing, covering or including the elements listed following the term, but not excluding other unrecited elements.

The term “consisting essentially of” means the list of elements may include additional components or excipients that do not materially contribute to the working of the invention.

The term “consisting of” is an exclusive term and means consisting only of.

The term “therapeutically effective amount” or “effective amount” means an amount that, when administered to an animal for treating a disease, disorder or condition, is sufficient to produce a desired therapeutic effect (e.g., to affect treatment for that disease).

The terms “composition” and “formulation” are synonymous and are used interchangeably herein.

1000 mL of 0.25% sodium dodecyl sulfate solution refers to a 1000 mL solution containing 2.5 g of sodium dodecyl sulfate.

The terms “dissolution” and “dissociation” are synonymous and are used interchangeably herein.

The term “PVP” refers to polyvinylpyrrolidone, also known as povidone. The term “PVPK30” refers to polyvinylpyrrolidone K30.

The term “RPM” refers to rotations per minute.

The term “FNF” refers to fenofibrate.

The term “hypromellose” (INN), is short for hydroxypropyl methylcellulose (HPMC), which is a semisynthetic, inert, viscoelastic polymer used in eye drops, as well as an excipient and controlled-delivery component in oral medicaments, found in a variety of commercial products.

The term “HPC” refers to hydroxypropyl cellulose, which is an ether of cellulose where some of the hydroxyl groups of the cellulose have been hydroxypropylated forming —OCH₂CH(OH)CH₃ groups. Lacrisert is a formulation of hydroxypropyl cellulose that is used for artificial tears. Solubility of HPC in water is temperature dependent, it is readily soluble at temperatures below the cloud-point (the temperature below which the polymer starts to phase-separate, and two phases appear, thus becoming cloudy), which is around 45° C. HPC is available commercially in different viscosity grades, with an average molecular weight (MW) grades ranging from 20 to 1500 kDa. The low MW grades most typically are used as binders (Table 2). HPC is a superior binder and has shown equivalent binder efficiency and good compactibility when added as a solution or in dry powdered form (Skinner & Harcum, 1998). Additionally, for dry addition, fine particle size grades are preferred because of faster hydration and uniformity of mixing and distribution. The coarse grades are preferred for solution addition because they disperse more easily without lumping.

### EXAMPLES

The invention will now be described in more detail with reference to the following non-limiting examples.

### Example 1: Production of a Fenofibrate Composition from an Acetone Solution

A fenofibrate composition was produced with an acetone solution of fenofibrate, cross-linked povidone and cross-linked sodium carboxymethyl cellulose according to the following steps:
a) 8 kg of fenofibrate was dissolved in 12 L of acetone;
b) 8 kg of cross-linked povidone, 9.5 kg of cross-linked sodium carboxymethyl cellulose, 5 kg of lactose and 1 kg of sodium dodecyl sulfate were placed in a fluid bed and heated to 30° C.;
c) the fenofibrate acetone solution of step a) was sprayed into the fluid bed containing the cross-linked povidone, cross-linked sodium carboxymethyl cellulose, lactose and SDS mixture;
d) 1000 g of PVPK30 (polyvinylpyrrolidone) in 20.75 L of water was sprayed into the fluid bed as a binder in order to form granules;
e) the granules obtained from step d) were mixed with magnesium stearate; and
f) the granules of step e) were compressed into tablets.

The fenofibrate composition made by fenofibrate acetone solution was then tested for dissolution and the results are shown in Table 1.

TABLE 1
Fenofibrate dissolution rate comparison between 160 mg tablet of the invention
produced using acetone solution with LIPANTHYL ™ 160 mg lot no. 26937/160 mg

| | Rate of FNF dissolution in 1000 mL of pure water containing | | | |
|---|---|---|---|---|
| | 3.6 g of SDS at 50 RPM | | 1.8 g of SDS at 50 RPM | |
| | | Composition of | | Composition of |
| | LIPANTHYL ™ | the invention of | LIPANTHYL ™ | the invention of |
| Minutes | 160 mg | Example 1/160 mg | 160 mg | Example 1/160 mg |
| 30 | 55.2% | 52.5% | 11.5% | 14.3% |
| 90 | 64.5% | 60.1% | 12.2% | 14.3% |

### Example 2: Production of a 145 mg Fenofibrate Composition from an Acetone Solution Containing 1 Part of Organic Acid and 1 Part of Fenofibrate

a) 2.9 kg of fenofibrate and 2.9 kg of tartaric acid were dissolved in 18 L of acetone;
b) 2.9 kg of cross-linked povidone, 2.9 kg of cross-linked sodium carboxymethyl cellulose and 0.6 kg of sodium dodecyl sulfate were placed in a fluid bed and heated to 30° C.;
c) the fenofibrate and tartaric acid acetone solution of step a) was sprayed into the fluid bed containing the cross-linked povidone, cross-linked sodium carboxymethyl cellulose and SDS mixture;
d) 400 g of PVPK30 (polyvinylpyrrolidone) was dissolved in 4.6 L of water, and the resulting solution was sprayed into the fluid bed as a binder to form granules;
e) the granules obtained from step d) were mixed with magnesium stearate; and
f) the granules of step e) were compressed into tablets.

The fenofibrate compositions made by fenofibrate acetone solution were then tested for amount of dissolution: the dissolution amount of fenofibrate was 55 mg for a 145 mg fenofibrate tablet after a dissolution test at 60 minutes in 1000 mL of neutral water containing 2.5 g of sodium dodecyl sulphate stirring at 75 RPM.

### Example 3: Production of a 120 mg Fenofibrate Tablet Composition from an Acetone Solution Containing 1 Part of Fenofibrate and 2 Parts of Malic Acid

a) 2.4 kg of fenofibrate and 4.8 kg of malic acid were dissolved in 36 L of acetone;
b) 2.4 kg of cross-linked povidone, 2.4 kg of cross-linked sodium carboxymethyl cellulose and 0.56 kg of sodium dodecyl sulfate were placed in a fluid bed and heated to 30° C.;
c) the fenofibrate and malic acid acetone solution of step a) was sprayed into the fluid bed containing the cross-linked povidone, cross-linked sodium carboxymethyl cellulose and SDS mixture;
d) 400 g of PVPK30 (polyvinylpyrrolidone) was dissolved in 4.6 L of water, and the resulting solution was sprayed into the fluid bed as a binder to form granules;
e) the granules obtained from step d) were mixed with magnesium stearate; and
f) the granules of step e) were compressed into tablets.

The fenofibrate compositions made by fenofibrate acetone solution were then tested for dissolution resulting in a fenofibrate dissolution amount of 54.3 mg for a 120 mg fenofibrate tablet after a dissolution test at 60 minutes in 1000 mL of neutral water containing 2.5 g of sodium dodecyl sulphate stirring at 75 RPM.

### Example 4: Production of a 90 mg Fenofibrate Tablet Composition from an Acetone Solution Containing 1 Part of Fenofibrate and 5 Parts of Citric Acid

a) 2.7 kg of fenofibrate and 13.5 kg of citric acid were dissolved in 31.2 L of acetone and 31.2 kg of ethanol together;
b) 2.25 kg of cross-linked povidone, 2.25 kg of cross-linked sodium carboxymethyl cellulose and 0.84 kg of sodium dodecyl sulphate were placed in a fluid bed and heated to 30° C.;
c) the fenofibrate and citric acid acetone solution of step a) was sprayed into the fluid bed containing the cross-linked povidone, cross-linked sodium carboxymethyl cellulose and SDS mixture;
d) 600 g of PVPK30 (polyvinylpyrrolidone) was dissolved in 6.9 L of water, and the resulting solution was sprayed into the fluid bed as a binder to form granules;
e) the granules obtained from step d) were mixed with magnesium stearate; and
f) the granules of step e) were compressed into tablets.

The fenofibrate compositions made by fenofibrate acetone solution were then tested for dissolution resulting in a fenofibrate dissolution amount of 54.6 mg for a 90 mg fenofibrate tablet after a dissolution test at 120 minutes in 1000 mL of neutral water containing 2.5 g of sodium dodecyl sulphate stirring at 75 RPM.

### Example 5: Bioequivalence Study of a 160 mg Fenofibrate Tablet Composition Produced Using Acetone Solution Against LIPANTHYL™ 160 mg Tablet

The 160 mg fenofibrate (III) composition of the invention of Lot S190924, as described herein, was compared with 160 mg LIPANTHYL™ tablets of Recipharm Fontaine (France) manufactured by ABBOTT LABORATORIES (Singapore) Private Limited (Lot 28782) through administration to 51 normal healthy adult volunteers in a bioequivalence study.

The bioequivalence study was an open-label, single-dose, randomized, two-period, two-sequence, crossover study under fasted and fed conditions, performed in Queen Mary Hospital Phase I Clinical Trials Centre (HKU Phase 1 Clinical Trials Centre) and analyzed by the Pharmacokinetics and Statistical Team of the Clinical Trials Centre, The University of Hong Kong.

Each qualified subject participated in a Screening Period (28 days), and was assigned into two groups (Group TR or Group RT with T symbolized as Test product: Lot S190924 herein while R symbolized as Reference product: Recipharm Fontaine's (France) LIPANTHYL™ tablet) with two treatment periods (Group TR received Test product in Period 1 and Reference product in Period 2 while Group RT received Reference product in Period 1 and Test product in Period 2). Periods 1 and 2 were separated with a minimum washout period of 7 days. In the Fasted Study, subjects were fasted for at least 10 hours at night before the time of dosing in the following morning. In the Fed Study, subjects were fasted for at least 10.5 hours and followed by a high-calorie and high fat test meal taken within 30 minutes, followed by swallowing the whole study drug at 30 minutes from the start of the meal. The pharmacokinetic (PK) blood sampling was collected within 2 hours prior to drug administration, 0.5 h, 1 h, 1.5 h, 2 h, 2.5 h, 3 h, 3.5 h, 4 h, 4.5 h, 5 h, 5.5 h, 6 h, 6.5 h, 7.5 h, 12 h, 24 h, 48 h, and 72 h after study drug administration (19 samples per pharmacokinetic profile) in each period of Parts 1 or 2. At each PK sampling time point, 4 mL PK blood samples were collected into PET vacutainer tubes containing K2EDTA, and placed in an ice bath until the samples were centrifuged to obtain plasma, and shortly after stored at ≤−70° C. for analysis. After absorption following oral administration, fenofibrate is quickly metabolized to the active metabolite fenofibric acid. The terminal half-life of fenofibric acid varies at about 20 hours. As unmetabolized fenofibrate cannot be detected in the plasma following oral intake, the active metabolite (fenofibric acid), which provides the therapeutic effect of fenofibrate, was used to demonstrate bioequivalence. Hence, blood samples were used to determine the concentration of fenofibric acid in plasma by a validated UPLC-MS/MS method.

The test product is considered to be bioequivalent with a reference product when the 90% confidence intervals (CI) for the ratio of the geometric means of Cₘₐₓ, AUC₀₋ₜ and AUC_{0-∞} were within the predetermined equivalent range of 80.00-125.00%.

As a result, 25 randomized subjects completed the Fasted Study and 26 randomized subjects completed the Fed Study.

Results of bioequivalence of the Fasted Study are summarized in Table 2.

TABLE 2
Summary of bioequivalence in the Fasted Study

| | Geometric mean and ratio (N = 26) | | | Intra- | | |
|---|---|---|---|---|---|---|
| PK Parameters | Test | Reference | T/R | subject | | Power |
| (Unit) | Product (T) | Product | (%) | CV % | 90% CI | % |
| Cₘₐₓ (μg/mL) | 3.76 | 3.87 | 97.22 | 27.28 | 85.45-110.61 | 76.17 |
| AUC₀₋ₜ (μg · h/mL) | 107.53 | 113.04 | 95.13 | 12.72 | 89.47-101.15 | 99.91 |
| AUC_{0-∞} (μg · h/mL) | 134.19 | 144.73 | 92.71 | 14.34 | 86.38-99.51 | 97 |

The corresponding 90% CIs of Cₘₐₓ, AUC₀₋ₜ, and AUC_{0-∞} for the test/reference geometric mean ratios were 85.45-110.61%, 89.47-101.15% and 86.38-99.51%, respectively. All data fell within 80.00% and 125.00%. A sensitivity analysis was performed for the BE analysis of AUC₀₋ₜ and AUC_{0-∞} excluding subjects with AUC₀₋ₜ/AUC_{0-∞}<80%. The statistical results showed that the bioequivalence of the test and reference products was still valid in the Fasted Study.

The results of bioequivalence of the Fed Study are summarized in Table 3.

TABLE 3
Summary of bioequivalence in the Fed Study

| | Geometric mean and ratio (N = 26) | | | Intra- | | |
|---|---|---|---|---|---|---|
| PK Parameters | Test | Reference | T/R | subject | 90% CI | Power |
| (Unit) | Product (T) | Product | (%) | CV % | (%) | % |
| Cₘₐₓ (μg/mL) | 11.38 | 12.15 | 93.67 | 7.94 | 90.21-97.26 | 100 |
| AUC₀₋ₜ (μg · h/mL) | 186.36 | 191.41 | 97.36 | 8.99 | 93.31-101.60 | 100 |
| AUC_{0-∞} (μg · h/mL) | 201.49 | 205.97 | 97.82 | 8.93 | 93.77-102.05 | 100 |

The mean plasma drug concentration-time curve after the administration of test and reference products in the Fed Study is shown in FIG. 2.

The corresponding 90% CIS of Cₘₐₓ, AUC₀₋ₜ, and AUC_{0-∞} for the test/reference geometric mean ratios were 90.21-97.26%, 93.31-101.60% and 93.77-102.05%, respectively. All data fell within 80.00% and 125.00%.

The data indicates that the test and reference fenofibrate tablets (III) are bioequivalent when administered under fasted and fed conditions. Safety data indicates that the test product 160 mg fenofibrate tablets (III) given under fasted and fed conditions had no safety issue.

Any reference to or discussion of any document, act or item of knowledge in this specification is included solely for the purpose of providing a context for the present invention. It is not suggested or represented that any of these matters or any combination thereof formed at the priority date part of the common general knowledge, or was known to be relevant to an attempt to solve any problem with which this specification is concerned.

It is to be understood that various other modifications and/or alterations may be made without departing from the spirit of the present invention as outlined herein.

1. A pharmaceutical composition comprising fenofibrate, a water soluble excipient selected from a group comprising an organic acid, wherein the organic acid is selected from the group comprising malic acid, maleic acid, succinic acid, tartaric acid, citric acid and ascorbic acid; hydroxypropyl methyl cellulose (HPMC) and/or hydroxypropyl cellulose, and wherein the composition is produced by dissolving the fenofibrate and the water soluble excipient in ketone which is subsequently mixed with an expander and filler, wherein the composition produced is in the form of pellets, capsules, granules or tablets.

2. The pharmaceutical composition of claim 1, wherein the expander and filler of the composition comprises cross-linked povidone, cross-linked carboxymethyl sodium cellulose, lactose, sugar, microcrystalline cellulose, a small amount of povidone and sodium dodecyl sulfate, wherein the composition is in the form of granules, tablets or capsules.

3. The pharmaceutical composition of claim 1, wherein the fenofibrate composition comprises 1 part of fenofibrate and 1 to 6 parts of an organic acid which is selected from the group comprising malic acid, tartaric acid, maleic acid, succinic acid, fumaric acid, ascorbic acid and citric acid, wherein the composition is in the form of granules, tablets or capsules.

4. The pharmaceutical composition of claim 1, wherein the fenofibrate composition comprises fenofibrate, an organic acid selected from the group comprising malic acid, tartaric acid, maleic acid, succinic acid, ascorbic acid and citric acid, wherein the composition further comprises a carbonate salt selected from calcium carbonate, sodium bicarbonate and potassium bicarbonate, and wherein the composition is in the form of tablets.

5. The pharmaceutical composition of claim 1, wherein the fenofibrate composition comprises 1 part of fenofibrate and 1 to 6 parts of an organic acid which is selected from the group comprising malic acid, tartaric acid, maleic acid, succinic acid, ascorbic acid and citric acid; wherein the composition further comprises cross-linked povidone, cross-linked carboxymethyl sodium cellulose and sodium dodecyl sulfate, wherein the composition is in the form of granules, tablets or capsules.

6. The pharmaceutical composition of claim 1, wherein the composition comprises fenofibrate, a water soluble excipient including an organic acid selected from the group comprising malic acid, maleic acid, succinic acid, tartaric acid, ascorbic acid and citric acid, wherein the composition further comprises HPMC, HPC, cross-linked povidone and cross-linked carboxymethyl sodium cellulose.

7. (canceled)

8. (canceled)

9. (canceled)

10. (canceled)

11. (canceled)

12. The pharmaceutical composition of claim 1, wherein the composition comprises fenofibrate, malic acid, cross-linked povidone, cross-linked carboxymethyl sodium cellulose, wherein the composition is in the form of granules, tablets or capsules.

13. The pharmaceutical composition of claim 1, wherein the composition comprises fenofibrate, maleic acid, cross-linked povidone, cross-linked carboxymethyl sodium cellulose, wherein the composition is in the form of granules, tablets or capsules.

14. The pharmaceutical composition of claim 1, wherein the composition comprises fenofibrate, tartaric acid, cross-linked povidone, cross-linked carboxymethyl sodium cellulose, wherein the composition is in the form of granules, tablets or capsules.

15. The pharmaceutical composition of claim 1, wherein the composition comprises fenofibrate, succinic acid, cross-linked povidone, cross-linked carboxymethyl sodium cellulose, wherein the composition is in the form of granules, tablets or capsules.

16. The pharmaceutical composition of claim 1, wherein the composition comprises fenofibrate, citric acid, cross-linked povidone, cross-linked carboxymethyl sodium cellulose, wherein the composition is in the form of granules, tablets or capsules.

17. The pharmaceutical composition of claim 1, wherein the composition comprises fenofibrate, an organic acid which is selected from the group comprising malic acid, tartaric acid, maleic acid, succinic acid, fumaric acid, and citric acid, HPMC, HPC and a core comprising sugar and/or microcrystalline cellulose, wherein the composition is in the form of spheres.

18. The pharmaceutical composition of claim 1, wherein the composition comprises fenofibrate, an organic acid which is selected from the group comprising malic acid, tartaric acid, maleic acid, succinic acid, fumaric acid, and citric acid, HPC and a core comprising sugar and/or microcrystalline cellulose, wherein the composition is in the form of spheres.

19. The pharmaceutical composition of claim 1, wherein the composition comprises fenofibrate, an organic acid which is selected from the group comprising malic acid, tartaric acid, maleic acid, succinic acid, fumaric acid, and citric acid, HPMC and a core comprising sucrose and/or microcrystalline cellulose, wherein the composition is in the form of spheres.

20. The pharmaceutical composition of claim 1, wherein the composition comprises fenofibrate, an organic acid which is selected from the group comprising malic acid, tartaric acid, maleic acid, succinic acid and citric acid, calcium carbonate, cross-linked povidone, cross-linked carboxymethyl sodium cellulose, wherein the composition is in the form of tablets.

21. A method of preparing a fenofibrate composition comprising the steps of:
a) forming a fenofibrate solution by dissolving an amount of fenofibrate, hydroxypropyl methyl cellulose (HPMC), and hydroxypropyl cellulose (HPC) in an organic solvent, wherein the solvent is selected from acetone, butanone or pentanone;
b) optionally dissolving or dispersing an organic acid into the fenofibrate solution of step a), wherein the organic acid is selected from the group comprising malic acid, maleic acid, succinic acid, tartaric acid, fumaric acid, ascorbic acid and citric acid;
c) spraying the fenofibrate solution or dispersion of step a) or b) into a fluid bed or granulator containing an expander and filler to form a powder mixture;
d) the powder mixture of step c) is glued together to form granules using povidone (PVPK30), HPMC or HPC, and the granules are dried in the fluid bed or an oven; and
e) the granules of step d) are compressed into a tablet or filled into a capsule or a granules bag.

22. Use of preparation method of claim 1 in the manufacturing of a medicament for the treatment of hyperlipidemia or hypercholesterolemia in a patient, wherein less than 160 mg of fenofibrate is administered to the patient once daily.

23. Use of the composition of claim 21, in the manufacture of a medicament for treatment of hyperlipidemia or hypercholesterolemia in a patient, wherein less than 160 mg of fenofibrate is to be administered to the patient once daily.

24. (canceled)
